(19) United States
(12) Patent Application Publication
(10) Pub. No.: US 2021/0311046 A1
(43) Pub. Date: Oct. 7, 2021

(54) METHODS OF REDUCING INTERFERENCE IN IMMUNOASSAYS

(71) Applicants: Mark A. Zaydman, St. Louis, MO (US); Jonathan R. Brestoff Parker, St. Louis, MO (US); Ann Marie Gronowski, St. Louis, MO (US)

(72) Inventors: Mark A. Zaydman, St. Louis, MO (US); Jonathan R. Brestoff Parker, St. Louis, MO (US); Ann Marie Gronowski, St. Louis, MO (US)

(73) Assignee: Washington University, St. Louis, MO (US)

(21) Appl. No.: 17/224,576

(22) Filed: Apr. 7, 2021

Related U.S. Application Data
(60) Provisional application No. 63/006,395, filed on Apr. 7, 2020.

(51) Int. Cl. G01N 33/557; G01N 33/543

(52) U.S. Cl. CPC G01N 33/557; G01N 33/54373

(57) ABSTRACT

Among the various aspects of the present disclosure is the provision of a method of reducing interference (e.g., the Hook effect) in an immunoassay (e.g., a single-step homogeneous turbidometric or nephelometric immune assay).

### CROSS-REFERENCE TO RELATED APPLICATIONS

This application claims priority from U.S. Provisional Application Ser. No. 63/006,395 filed on 7 Apr. 2020, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD

The present disclosure generally relates to improving immunoassays.

### SUMMARY

Among the various aspects of the present disclosure is the provision of a method of reducing interference (e.g., the Hook effect) in an immunoassay (e.g., a single-step homogeneous turbidometric or nephelometric immune assay).

An aspect of the present disclosure provides for a method of reducing interference in an immunoassay. Another aspect of the present disclosure provides for a method of monitoring immunoassay reaction kinetics. Another aspect of the present disclosure provides for a method of detecting and correcting antigen excess. Another aspect of the present disclosure provides for a method of extending the analytical measurement range (AMR). In some embodiments, the method comprises generating, providing, or having been provided a target analyte concentration vs. time curve. In some embodiments, the method comprises measuring an area under the curvature (AUCU). In some embodiments, the curve is generated by: (i) providing or having been provided a sample comprising a target analyte; (ii) contacting the sample comprising the target analyte with antibodies capable of crosslinking the target analyte to form an immune complex; and/or (iii) detecting the target analyte and plotting the absorbance vs. time. In some embodiments, the sample is a biological sample comprising a target analyte from a subject. In some embodiments, the AUCU provides a log-linear calibration curve and/or increases proportionally to the target analyte concentration above a limit of an analytical measurement range (AMR) of a reaction endpoint. In some embodiments, the target analyte concentration comprises measuring absorbance or light scattering of the immune complexes. In some embodiments, measuring the AUCU comprises: (a) normalizing absorbance versus time data resulting in a normalized kinetic data function; and/or (b) calculating the AUCU as a sum of the difference between the normalized kinetic data function and a line of unity, wherein the line of unity is the line resulting from the normalized absorbance at t=0 and t=tend, wherein tend is the time at the reaction endpoint. In some embodiments, the target analyte in the sample is performed using an automated chemistry analyzer to monitor a formation of light-scattering immune complexes that are generated when the target analyte cross-links a target analyte-specific reagent antibodies or antibody coated beads. In some embodiments, the method of claim 1 is used if above the limit of the AMR, and a standard reaction endpoint calibration curve is used if below the limit of the AMR. In some embodiments, a calibration curve choice is automated via a software tool. In some embodiments, measuring the absorbance comprises measuring changes in light absorbance or light scattering. In some embodiments, measuring absorbance vs. time is performed by recording, via a computer, kinetic data by monitoring the reaction at regular intervals prior to the reaction endpoint. In some embodiments, the interference that is being reduced is the Hook effect. In some embodiments, the immunoassay is a single-step homogeneous turbidometric or light absorbance assay. In some embodiments, the immunoassay is a nephelometric or light scattering immune assay. In some embodiments, sample dilution is not required if there is antigen excess. In some embodiments, the AMR is extended by at least about 2-fold, at least about 3-fold, at least about 4-fold, at least about 5-fold, at least about 6-fold, at least about 7-fold, at least about 8-fold, at least about 9-fold, or at least about 10-fold. In some embodiments, the AMR is extended by at least about 10-fold. In some embodiments, the AUCU detects antigen excess. In some embodiments, the AUCU provides a second calibration curve for use in a zone of antigen excess. In some embodiments, the method quantifies high antigen concentrations without sample dilution.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1. A simple kinetic model predicts behavior of the κ sFLC immunoassay. (A) Kinetic model of a generic homogeneous turbidimetric immunoassay. (B) Measured κ sFLC concentration via standard end point absorbance change is plotted vs the true κ sFLC concentration. (C) Absorbance changes vs time point at 4 different κ concentrations. (D) The kinetic traces in (C) were baseline subtracted and normalized to the end point signal change. Black lines/symbols=data; dashed red lines=model predictions.

FIG. 2. The AUCU extends the AMR in silico. The model from FIG. 1A was used to simulate the κ sFLC reaction for various antigen concentrations. (A) Concentrations of the indicated species (top) and the normalized [AbAgAb] (bottom) are plotted vs time point under conditions of antibody excess (left, [Ag] =1 mg/dL) or antigen excess (right, [Ag]=200 mg/dL). (B) Simulated dose-response curves for the end point [AbAgAb] (dashed) or the AUCU (solid) vs Ag concentration.

FIG. 3. The AUCU provides a calibration curve in the zone of antigen excess. The initial result (A and D) antigen excess factor (B and E) and AUCU (C and F) are plotted vs final concentration for 144 κ sFLC samples (A-C) or 27 RF samples (D-F). Large open symbols indicate samples that were repeated because of built-in antigen excess detection. Small closed symbols indicate the samples that were measured only at the initial dilution. The solid lines in (A) and (C) represent the lines of unity. The dotted line in (B) represents the threshold value of 75, below which antigen excess is likely (7). The solid lines in (C) and (F) show the fit of the indicated logarithmic functions to the results from the repeated samples. The gray shaded symbols in (D-F) indicate 3 dilutions (1×, 5×, 10×) of a single high RF activity sample (451 IU/mL).

FIG. 4. Overview of the genetic algorithm. An initial population of 500 individual solutions was generated by randomly sampling a uniform distribution spanning the boundaries indicated in TABLE 1 for each of the six free parameters: k1, k2, k3, k4, Ab0, and delABS. The individual solutions were then numerically simulated for four different antigen concentrations using a time-adaptive 4th order Runge-Kutta method. The error of each solution was evaluated using a sum of squares difference between the simulated traces and the experimental data from the 4 dilutions of a single patient sample measured using the kappa sFLC assay (FIG. 10). Random pairs of “mates” were selected from the 50 lowest error solutions and offspring solutions were generated by randomly selecting one of the two parent parameter values to pass to the offspring for each free parameter. In addition, the ten lowest error solutions were cloned to the next generation. Next, the offspring were subjected to a stochastic mutation process where each parameter had a 20 percent chance of being chosen for mutation and, if selected, a new parameter value was generated by randomly sampling a uniform distribution spanning the current value +/−25%. In this way, a new generation of 500 solutions was generated. The entire process was repeated for 500 total generations to allow the evolution of a “best-fit” individual.

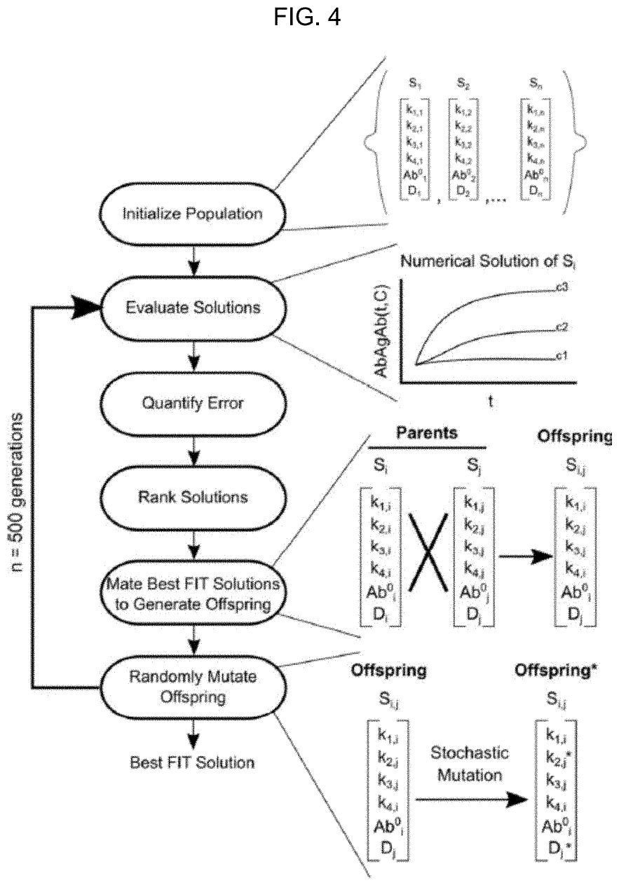

FIG. 5. Evolution of the best-fit solution using the genetic algorithm. The free-parameter values (k1, k2, k3, k4, Ab0) and sum-of-squares error (error) of the best-fit individual are plotted versus generation number.

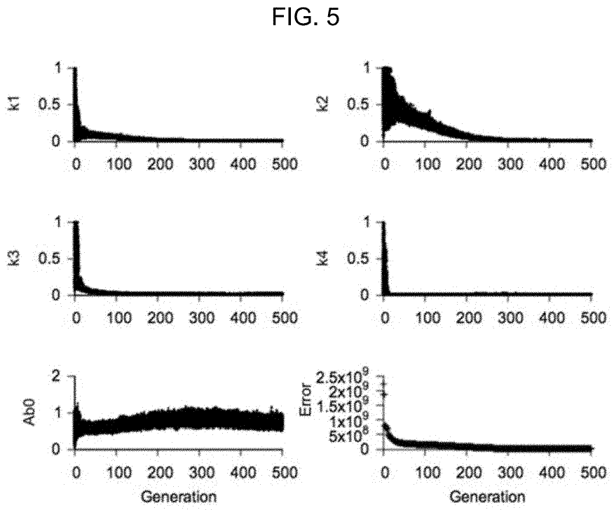

FIG. 6. Kinetics of the kappa sFLC assay. (A) The reaction absorbance is plotted versus timepoint for 144 clinical kappa sFLC measurements. (B) The traces in panel A were normalized, as described in the methods, and plotted versus timepoint.

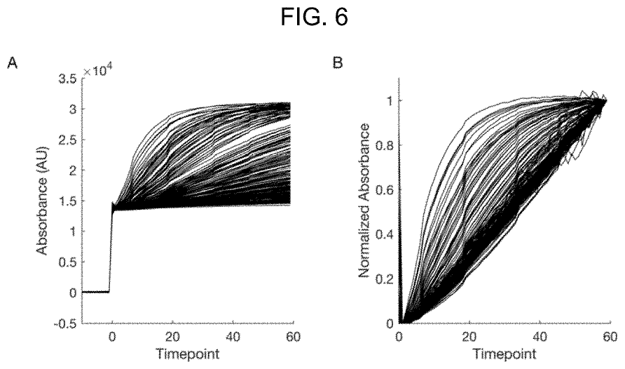

FIG. 7. Kinetics of the RF assay. (A) The reaction absorbance is plotted versus timepoint for the 29 clinical samples that contained RF activity above the manufacturers analytical measuring range. (B) The traces in panel A were normalized, as described in the methods, and plotted versus timepoint.

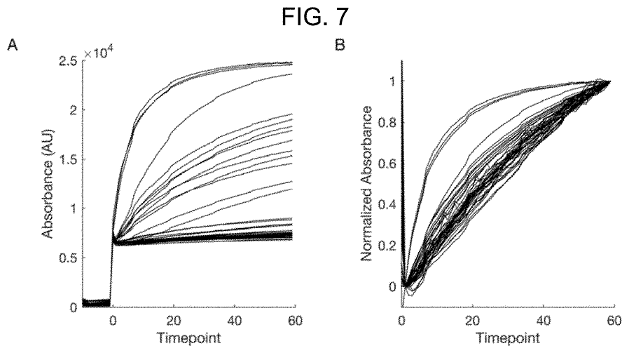

FIG. 8. The impact of assay duration on the dose-response curve. The parameterized model is described in FIG. 1 was simulated over a wide range of antigen concentrations. (A) The concentration of light-scattering complexes, [AbAgAb], is plotted versus timepoint for four different antigen concentrations. (B) The endpoint [AbAgAb] is plotted versus antigen concentration using a reaction ending timpoint of 5, 10, 20, 40, or 63.

FIG. 9. The AUCU method of estimating curvature is less sensitive to noise than a point estimate. (A) Raw change in absorbance versus timepoint is plotted for a single RF measurement. (B) Simulated curve generated by adding random Gaussian noise to the trace in panel A to achieve a specific signal-to-noise ratio (SNR). (C) The AUCU and a point estimate of curvature based upon the ratio between late and early absorbance changes (ΔΔ Absorbance), i.e. the “Antigen-Excess Factor” described by Urdal and Colleagues (7), was calculated for 1000 simulated traces at each SNR. The coefficient of variation (CV) is plotted versus SNR.

### DETAILED DESCRIPTION OF THE INVENTION

The present disclosure is based, at least in part, on the derivation of a kinetic parameter, the area under the curvature (AUCU), which increases proportionally to analyte concentration well beyond the established AMR. As shown herein, this novel analysis uses data that is already routinely collected, thus the AUCU method can be easily adapted to any hospital or reference laboratory system to decrease costs associated with repeat testing. The disclosed methods are attractive because it will decrease the potential for falsely low results and extend the analytical measuring range simply through a software application.

The turbidimetric homogeneous immunoassay represents a flexible clinical testing platform that offers short assay time and the potential for full automation. A significant limitation of this assay format is nonlinearity in the setting of antigen excess, i.e., the hook effect. Conventional methods for correcting antigen excess involve sample dilution and repeated measurement-steps that introduce additional time, costs, and opportunities for laboratory errors. In this study, a novel kinetic analysis method is developed that allows accurate quantification in the setting of antigen excess without requiring sample dilution.

The problem solved by the presently disclosed invention is that, only within a limited analytical measurement range (AMR), the concentration of detectable immune complexes increases in proportion with analyte concentration, allowing for accurate quantification. Above the AMR, the concentration of analytes exceeds the concentration of reagent antibodies (antigen-excess) causing the antibodies to become saturated with the analyte and preventing the antibody cross-linking step required for immune-complex formation. As a result, above the AMR, the measured signal will paradoxically decrease with further increases in analyte concentration leading to underestimation of the true concentration or, in worst case scenarios, false negative results. Errors caused by antigen-excess must be avoided as they can negatively impact patient care. Established strategies for dealing with antigen-excess involve identifying potentially problematic samples, performing a dilution to target a concentration within the AMR, and repeating the measurement. Unfortunately, sample repetition wastes time and expensive reagents compromising the very features that make a single-step immunoassay an attractive assay format. Therefore, a limited AMR is associated with significant costs to the hospital system or reference laboratory due to repeat testing. Furthermore, the additional manipulation of the samples introduces new opportunities for clerical errors that may cause patient harm.

To solve this problem, as disclosed herein, is a new analysis methodology that extends the AMR to allow accurate quantification of samples with high analyte concentration without performing dilution or repeated measurement. The disclosed methodology can allow significant time and cost reduction in clinical laboratory testing.

The disclosed methodology provides a calibration curve that is only valid above the AMR. For samples below the AMR the standard endpoint calibration curve should be used. The decision regarding which calibration curve to use can be automated via a software tool. More specifically, a low value for the Area Under the Curvature (AUCU) indicates that a result within the AMR, determined using the standard calibration, is indeed valid. High Area Under the Curvature values or estimated values beyond the AMR obligate the use of the disclosed novel methodology. It is noted that plotting is not a requirement as the measurement of the AUCU can be fully automated.

Because multi-step immunoassays involve the use of a washing step in which unbound excess antigen is removed before application of a secondary antibody, antigen-excess is not usually a problem for multi-step (heterogeneous) immunoassays as they are for a single-step (homogeneous) immunoassays. However, the advantage of a homogeneous (single-step) immunoassay is that multiple reaction steps (primary antibody reaction, wash, secondary antibody reaction) are time-consuming, leading to increased machine time and longer result turn-around-time.

Single-step immunoassays are widely used in the clinical laboratory to quantify analytes in patient samples. Attractive features of a single-step immunoassay are short assay times and the potential for full-automation, both of which help to reduce costs and decrease laboratory turn-around-time. In these assays, the target analyte is detected using an automated chemistry analyzer to monitor the formation of light-scattering immune complexes that are generated when the analyte cross-links analyte-specific reagent antibodies or antibody coated beads. Within a limited analytical measurement range (AMR), the concentration of immune complexes increases in proportion with analyte concentration, allowing for accurate quantification. Above the AMR, the concentration of analytes exceeds the concentration of reagent antibodies (antigen-excess) causing the antibodies to become saturated with the analyte and preventing the antibody cross-linking step required for immune-complex formation. As a result, above the AMR, the measured signal will paradoxically decrease with further increases in analyte concentration leading to underestimation of the true concentration or, in worst case scenarios, false negative results. Errors caused by antigen-excess must be avoided as they can negatively impact patient care. Established strategies for dealing with antigen-excess involve identifying potentially problematic samples, performing a dilution to target a concentration within the AMR, and repeating the measurement. Unfortunately, sample repetition wastes time and expensive reagents compromising the very features that make a single-step immunoassay an attractive assay format. Therefore, a limited AMR is associated with significant costs to the hospital system or reference laboratory due to repeat testing. Furthermore, the additional manipulation of the samples introduces new opportunities for clerical errors that may cause patient harm. Here is disclosed a new analysis methodology that extends the AMR to allow accurate quantification of samples with high concentration without performing dilution or repeated measurement. Adaptation of our methodology would allow significant time and cost reduction in clinical laboratory testing.

Specifically, using kinetic modeling and empiric analysis of established high-volume laboratory tests, we derived a kinetic parameter, the area under the curvature (AUCU), which increases proportional to analyte concentration well beyond the established AMR. As this novel analysis uses data that is already routinely collected, the AUCU method can be easily adapted to any hospital or reference laboratory system to decrease costs associated with repeat testing. With real patient samples, we used the AUCU method to achieve greater than 10-fold extension of the AMR with real clinical tests.

Conventional Turbidimetry and Nephelometry

When particles are suspended in a solution in a cuvette, they make the solution unclear (turbid). Incident light entering the cuvette will be subjected to three reactions: 1-some of the light will be absorbed (blocked) by the particles; 2-some will be transmitted through the cuvette; and 3-some will be scattered in various directions.

Turbidimetry

Turbidimetry is involved with measuring the amount of transmitted light (and calculating the absorbed light) by particles in suspension to determine the concentration of the substance in question. Amount of absorbed light, and therefore, concentration is dependent on the number of particles, and size of particles. Measurements are made using light spectrophotometers

Clinical Applications

Determination of the concentration of total protein in biological fluids such as urine and CSF which contain small quantities of protein (mg/L quantities) using trichloroacetic acid. Determination of amylase activity using starch as substrate. The decrease in turbidity is directly proportional to amylase activity. Determination of lipase activity using triglycerides as substrate. The decrease in turbidity is directly proportional to lipase activity.

Nephelometry

Principle

Nephelometry is concerned with measurement of scattered light from a cuvette containing suspended particles in a solution. The components of a nephelometer are the same as a light spectrophotometer except that the detector is placed at a specific angle from the incident light. The detector is a photomultiplier tube placed at a position to detect forward scattered light. Detectors may be placed at 90°, 70° or 37° depending on the angle at which most scattered light is found. Because the amount of scattered light is far greater than the transmitted light in a turbid suspension, nephelometry offers higher sensitivity than turbidimetry. The amount of scattered light depends on the size and number of particles in suspension. For most clinical applications, the light source is a tungsten lamp giving light in the visible region. For higher sensitivity and for applications that determine the size and number of particles in suspension, laser light nephelometers are used.

Clinical Applications of Nephelometry.

Nephelometry can be used to determine concentrations of unknowns where there is antigen-antibody reactions such as: determination of immunoglobulins (total, IgG, IgE, IgM, IgA) in serum and other biological fluids; determination of the concentrations of individual serum proteins; hemoglobin, haptoglobin, transferring, c-reactive protein, al-antitrypsin, albumin (using antibodies specific for each protein); or determination of the size and number of particles (e.g., laser-nephelometer).

Considerations in Conventional Turbidimetry and Nephelometry

The reaction in turbidimetry & nephelometry does not follow Beer's Law, therefore, standard curves must be plotted and the concentration of the unknown is determined from the standard curve. Because the absorbance is dependent on both number and size of particles, the standard solution which is used for the standard curve must have similar size in suspension as unknown. Because some precipitation and settlement of particles may occur with time, in order to obtain good accuracy it is important to mix the sample well prior to placing the cuvette in the instrument and keep the same time for measurement of every sample throughout the measurement. Kinetic reactions (measurement of the progress of reaction with time) provides higher degree of accuracy, sensitivity, precision and less time than end-point reactions (e.g., measuring the reaction at the start and finish of the reaction). Additionally in kinetic reactions there is no need for reagent blank since the previous reading is taken as the base-line for the next reading. Kinetic reaction may be taken in, for example, 60, 90, or 120 seconds (taking readings at 10 seconds intervals, for example), whereas endpoint reactions may take much longer time e.g., 15-120 minutes.

Selection of a Wavelength

If both solution and suspended particles are colorless, then use any wavelength in the visible range. If the solution is colored but the particles are not colored, then use a wavelength that gives minimum absorption for the solution. If the particles are colored and the solution is colorless then use a wavelength that gives maximum absorption with the particles. If both solution and particles are colored then use two wavelengths; one that gives minimum absorbance for the solution and the other one maximum absorbance for the particles. Subtract the solution absorbance from the particles' absorbance.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Kinetic Approach Extends the Analytical Measurement Range and Corrects Antigen Excess in Homogeneous Turbidimetric Immunoassays

The following example describes a method for extending the analytic measurement range of immunoassays and reducing repeat testing by ameliorating the hook effect. Shown herein are novel methods that extend the AMR.

Homogeneous turbidimetric immunoassays are widely used in the clinical laboratory and offer short assay times, reduced reagent costs, and the potential for full automation. However, these assays have a limited analytical measurement range (AMR) above which antigen excess leads to falsely low estimates of the analyte concentration (i.e., the hook effect). Traditional methods for correction of antigen excess require sample dilution, compromising time, and cost-efficiency. Therefore, described here, is a novel method that extends the AMR.

Briefly, a kinetic model of a generic homogeneous turbidimetric immunoassay was built and then parameterized using a genetic algorithm. Kinetic features that could be used to extend the AMR were identified and subsequently validated with clinical data from consecutive measurements of 2 homogeneous turbidimetric immunoassays: κ serum free light chain and rheumatoid factor. A novel kinetic parameter, the area under the curvature (AUCU), was derived that increases in proportion to the analyte concentration in a range beyond the AMR of conventional end point methods. When applied to clinical data, the AUCU method provided a log-linear calibration curve in the zone of antigen excess extending the AMR by >10-fold for 2 different immunoassays. In summary, the AUCU method described herein detects and corrects antigen excess, extending the AMR in homogeneous turbidimetric immunoassays. The advantage of this method over conventional methods is a reduction in the number of repeated samples, resulting in significant time and cost savings.

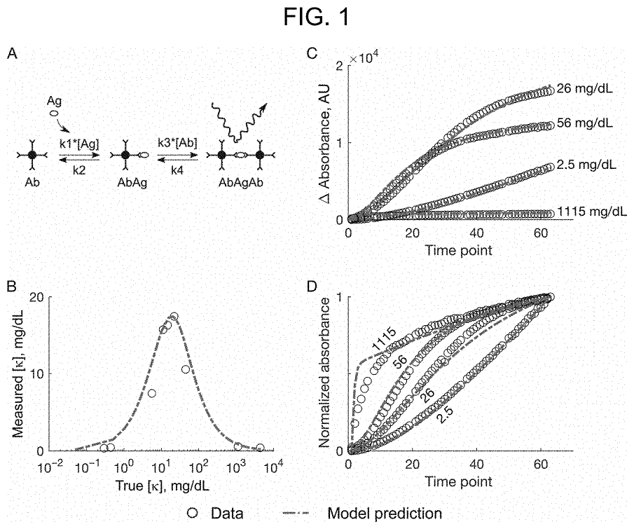

The homogeneous turbidimetric immunoassay is a widely used assay format that allows for rapid and fully automated detection of a variety of analytes in clinical samples. This simple assay involves adding an aliquot of a patient sample to a solution of antibodies or antibody-coated particles. If present, the target antigen (i.e., the analyte) will cross-link the antibodies, forming immune complexes that can be detected by monitoring changes in light absorbance (turbidimetry) or scattering (nephelometry) (FIG. 1A). Typically, the end point signal change is used to estimate the antigen concentration by assuming a pseudo-zero-order reaction rate so that the amount of immune complexes produced within a fixed time interval is linearly proportional to the antigen concentration. Because this assumption is valid only under conditions of antibody excess, significant errors can arise if the antigen concentration exceeds that of the antibodies. Under conditions of antigen excess, the end point signal paradoxically decreases with increasing antigen concentration, producing a bell-shaped response curve, i.e., the Heidelberger curve (1, 2). This behavior is commonly referred to as the hook effect. Errors introduced by antigen excess can harm patients by obscuring the detection of a pathologic state or the effectiveness of therapy (3-5). Because the end point absorbance changes produced under conditions of antigen excess can be indistinguishable from those produced under antibody excess, detection of antigen excess can be challenging.

Clinical spectrophotometers and nephelometers are capable of recording kinetic data by monitoring the reaction at regular intervals before the end point. These kinetic data are informative and have been used to help identify antigen excess when present (6-9). Many clinical instruments now carry built-in linearity and kinetic flags that identify potentially problematic samples that should be repeated after sample dilution. However, performing dilutions and repeat testing adds significant costs and inefficiencies to clinical laboratories. In addition, current kinetic flags do not detect all cases of antigen excess. Therefore, there is a need to identify novel methods that mitigate the problems associated with antigen excess in homogeneous turbidimetric immunoassays. Here, we formally describe area under the curvature (AUCU),³ a novel method to monitor immunoassay reaction kinetics. We also show that AUCU can be used to detect and correct antigen excess, extending the analytical measurement range (AMR) of 2 widely used immunoassays at least 10-fold.

Materials and Methods

Assays

First, κ serum free light chains (sFLCs) were quantified using the Freelite Human Kappa Free kit (The Binding Site Group; measuring range, 0.37-5.62 mg/dL) on the open channel of the Cobas c501 CHEMISTRY Analyzer (Roche Diagnostics). Rheumatoid factor (RF) was quantified using the Rheumatoid Factors II kit (Roche Diagnostics; measuring range, 10-130 IU/mL) on the Cobas c501. Kinetic data were downloaded daily from the instrument. Assays were performed according to manufacturer instructions.

Patient Samples

A single patient sample with a high κ sFLC concentration (4408 mg/dL) was used to generate a dose-response curve and to parameterize a kinetic model of a generic homogeneous turbidimetric immunoassay. Subsequently, the kinetic data from routine κ sFLC measurements in 150 consecutive clinical samples were collected for validation of the model predictions. Six samples had a κ sFLC concentration below the manufacturer's measurable range and were excluded from the analysis. To test the generalizability of the model predictions Across assays, the kinetic data from routine RF measurements in 133 consecutive clinical samples were collected. Of the samples, 106 had RF activities below the measurable range and were excluded from the analysis. Two additional dilutions of a single high RF activity sample were prepared to provide additional data at intermediate concentrations.

Kinetic Model Derivation and Fitting

A simple chemical reaction scheme involving 2 sequential elementary reactions was devised to model a generic homogeneous turbidimetric immunoassay (FIG. 1A). In the first step, a free antigen (Ag) complexes with a free antibody (Ab) to form a bimolecular complex (AbAg). In the second step, AbAg encounters a second Ab producing a trimolecular antibody-antigen-antibody (AbAgAb) complex. Assuming that only AbAgAb efficiently scatters light, the absorbance at any given time was taken to be linearly proportional to the [AbAgAb]. This model is described by the following set of differential equations (Eqs. 1-4), initial conditions (Eqs. 5-8), and conservation of mass equations (Eqs. 9 and 10).

Differential Equations

$$\begin{matrix}\frac{d [ Ab ]}{dt} = - k 1 [ Ab ] [ Ag ] + k 2 [ AbAg ] - k 3 [ Ab ] [ AbAg ] + k 4 [ AbAg ] & ( 1 ) \\ \frac{d [ Ag ]}{dt} = - k 1 [ Ab ] [ Ag ] + k 2 [ AbAg ] & ( 2 ) \\ \frac{d [ AbAg ]}{dt} = k 1 [ Ab ] [ Ag ] - ( k 2 + k 3 [ Ab ] ) [ AbAg ] + k 4 [ AbAgAb ] & ( 3 ) \\ \frac{d [ AbAgAb ]}{dt} = k 3 [ Ab ] [ AbAg ] - k 4 [ AbAgAb ] & ( 4 )\end{matrix}$$

where [X] indicates the concentration of species X, k1 and k2 are the forward and reverse rate constants for the first elementary reaction, respectively, and k3 and k4 are the forward and reverse rate constants for the second elementary reaction.

Initial Conditions

[Ab]₀=Ab₀ (5)

[Ag]₀=Ag₀ (6)

[AbAg]₀=0 (7)

[AbAgAb]₀=0 (8)

where subscript denotes the time point and t=0 is the time of the addition of the antigen to the reaction. Ab₀ and Ag₀ are the initial concentrations of free antibody and free antigen, respectively.

Conservation of Mass

[AbAg]₀=[Ab]+[AbAg]+2*[AbAgAb] (9)

[Ag]₀=[Ag]+[AbAg]+[AbAgAb] (10)

A genetic algorithm (10), i.e., a computer algorithm mimicking the principles of Darwinian evolution, was used to globally fit the model to the kinetic data from 4 different dilutions of a patient sample with high κ sFLC concentration. Briefly, an initial population of 500 solutions was generated by randomly sampling a broad parameter space. The solutions were solved numerically using the fourth order Runge-Kutta method with adaptive step-size control. The error of each solution was calculated using a sum of squared differences between the simulated curves and the experimental data. A pair of solutions were selected from the 50 solutions with the lowest error, and the parameters of the paired solutions were randomly combined to produce a new “offspring” solution. This “mating” process was repeated to generate a new generation of 490 offspring solutions. Next, the parameters of the offspring solutions were subjected to stochastic mutation with a probability rate of 20%. If selected for mutation, a new parameter value was selected by randomly sampling the range spanning ±25% of the current parameter value. Finally, the 10 best-fit solutions from the parent generation were copied forward to the offspring generation unchanged. In this way, the population of solutions was evolved for 500 generations. The entire process was repeated varying parameter search space windows, population sizes, and mutation rates to test the robustness of the evolved solution. Ultimately, the overall best-fit solution was selected.

Normalization

The kinetic absorbance vs time data were normalized according to the following equation:

$$NormAbs ( t ) = \frac{Abs ( t ) - Ab s ( 2 )}{Ab s ( end ) - Abs ( 2 )} \qquad ( 11 )$$

where Abs(t) is the absorbance at time point t, Abs(2) is the absorbance at the second time point following sample addition, and Abs(end) is the end point of the reaction. The second time point (t=2) was used for baseline subtraction to allow for the resolution of the sample-mixing artifact.

Calculation of the Antigen Excess Factor

The antigen excess factor was calculated as the ratio of the late and early absorbance changes as described previously by Urdal and colleagues (7).

$$Antigen Excess Factor = \frac{Delta Absorbance late}{Delta Absorbance early} = \frac{Abs ( 58 ) - Abs ( 50 )}{Abs ( 10 ) - Ab s ( 2 )} \qquad ( 12 )$$

Calculation of AUCU

The AUCU was calculated as the sum of the differences between the normalized kinetic data and the line connecting the initial and final normalized absorbance values, i.e., 0 and 1, respectively:

$$AUCU = {\sum }_{t = 1}^{N} ( NormAbs ( t ) - t / N ) \qquad ( 13 )$$

where NormAbs(t) is the normalized absorbance at time point t, and N is the total number of time points.

This study was approved by the Institutional Review Board at Washington University (protocol 201901012).

Results

The end point and kinetic behavior of the κ sFLC assay was studied using several dilutions of 1 serum specimen with an unusually high κ sFLC concentration (4408 mg/dL). At <10 mg/dL, the end point absorbance change increased with κ sFLC concentration (FIG. 1B and FIG. 10), consistent with the manufacturers reported linear range (0.37-5.62 mg/dL, package insert). At >10 mg/dL, the end point absorbance increase slowed before paradoxically decreasing because of antigen excess, resulting in the measured concentration grossly underestimating the true concentration. Notably, at all concentrations tested, the normalized absorbance vs time point traces exhibited progressively faster kinetics with increasing sFLC concentration (FIG. 1D), suggesting that kinetic analysis could be used to extend the AMR.

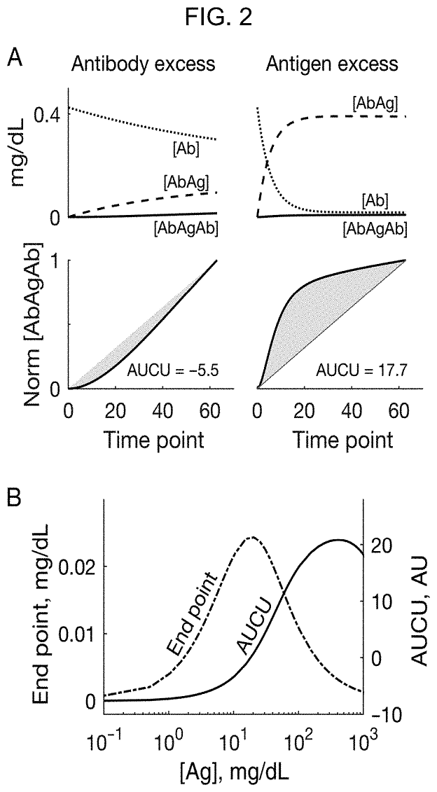

To explore the mechanisms underlying these kinetic behaviors, a minimal mathematic model of a general homogeneous turbidimetric immunoassay was derived using 2 sequential elementary reactions (FIG. 1A and Eqs. 1-10). Using a model-fitting algorithm that is based on the principles of natural evolution (10), the model was globally fit to the kinetic data from 4 different dilutions of the patient sample (FIG. 10; see also FIG. 1 and FIG. 2 and TABLE 1 and TABLE 2).

TABLE 1
Model parameters fit using genetic algorithm. Lower and upper bounds indicate
the parameter space sampled for the initial random population of solutions.
delAbs indicates the linear scaling factor that converts the fractional occupancy
of AgAbAb into an equivalent baseline-subtracted absorbance.

| | Unit | Lower Bound | Upper Bound | Best Fit Value |
|---|---|---|---|---|
| k1 | timepoint⁻¹ | 0.0001 | 1 | 0.0065774 |
| k2 | mg*dL⁻¹*timepoint⁻¹ | 0.0001 | 1 | 0.00782683 |
| k3 | timepoint⁻¹ | 0.0001 | 1 | 0.0147005 |
| k4 | mg*dL⁻¹*timepoint⁻¹ | 0.0001 | 1 | 0.00874191 |
| Ab₀ | mg/dL | 0.01 | 20 | 0.426249 |
| delABS | Unitless | 1 | 100,000 | 738043 |

TABLE 2
Best fit solutions for 9 additional runs of the genetic algorithm.

| Run | k1 | k2 | k3 | k4 | Ab0 | delAbs | Lack of Fitness |
|---|---|---|---|---|---|---|---|
| 1 | 0.0066 | 0.0068 | 0.0176 | 0.0065 | 0.5035 | 444916 | 17200000 |
| 2 | 0.0070 | 0.0075 | 0.0253 | 0.0043 | 0.4782 | 345155 | 16100000 |
| 3 | 0.0065 | 0.0050 | 0.0264 | 0.0035 | 0.4602 | 354112 | 15700000 |
| 4 | 0.0064 | 0.0035 | 0.0316 | 0.0011 | 0.4590 | 296388 | 15800000 |
| 5 | 0.0068 | 0.0077 | 0.0148 | 0.0070 | 0.5999 | 373516 | 16600000 |
| 6 | 0.0065 | 0.0069 | 0.0382 | 0.0070 | 0.2221 | 1060000 | 15700000 |
| 7 | 0.0068 | 0.0093 | 0.0224 | 0.0081 | 0.3219 | 840000 | 19600000 |
| 8 | 0.0065 | 0.0060 | 0.0311 | 0.0042 | 0.3828 | 438000 | 18000000 |
| 9 | 0.0068 | 0.0074 | 0.0300 | 0.0042 | 0.3580 | 505000 | 18300000 |
| avg | 0.0067 | 0.0067 | 0.0264 | 0.0051 | 0.4206 | 516901.3333 | 17004200 |
| std | 0.0002 | 0.0017 | 0.0073 | 0.0022 | 0.1117 | 257591.3456 | 1391538.758 |

The model reproduced both the end point and kinetic behaviors observed in the data (FIG. 1B-FIG. 1D, red dashed lines). The end point absorbance change demonstrated the bell-shaped response curve seen in the patient data (FIG. 1B) and, more generally, in all homogeneous turbidimetric immunoassays that use conventional end point analysis (5). Likewise, the kinetics of the simulated absorbance changes mirrored the experimental data (FIG. 10 and FIG. 1D). Specifically, at low antigen concentrations, the simulated and experimentally observed absorbance remained stationary for a short period before increasing with roughly linear kinetics, producing a positively curved trace. In contrast, at high antigen concentrations, both signals increased rapidly following sample addition and then slowed over time, creating a negative curvature.

The model was used to simulate the assay responses over a wide range of antigen concentrations (10⁻¹ to 10³) providing detailed mechanistic insights into how the simple reaction scheme gives rise to such complex kinetic changes in optical absorbance (FIG. 2). For low antigen concentrations (FIG. 2A, antibody excess), the free antibody concentration, [Ab], remained relatively constant throughout the reaction. As a result, the overall reaction rate is approximately zero order following a short delay that represents the time required to form the AbAg intermediate, which precedes formation of the light-scattering AbAgAb complex. Under conditions of antigen excess (FIG. 2A, antigen excess), the model predicts very different behavior. With an abundance of free antigen, Ag, mass action quickly converts Ab to AbAg, reducing the duration of the initial delay in absorbance change and producing an exponential decrease in [Ab]. Because the second step of the reaction scheme is dependent on [Ab], the rate of conversion of AbAg to AbAgAb decays over time, producing a progressively slower increase in [AbAgAb] (negative curvature) and limiting end point [AbAgAb] (hook effect). Although it could be challenging to distinguish conditions of antibody excess and antigen excess based on their end point [AbAgAb] alone, the positive vs negative curvature of their respective normalized [AbAgAb] vs time curves readily identified antibody excess and antigen excess, respectively. To quantitatively estimate the overall degree of curvature of these traces, we defined a derived parameter, the AUCU, as the sum of the finite differences between the normalized [AbAgAb] trace and the line connecting the initial and end point normalized [AbAgAb] occupancy, i.e., 0 and 1. The AUCU method was chosen among several methods of estimating curvature because it is mathematically simple to compute and it does not rely on the identification of a specific time point at which the curvature is informative. In silico, AUCU demonstrated an approximately log-linear relationship to sFLC concentrations between 10 and 100 mg/dL (FIG. 2B), suggesting that this derived kinetic parameter could be used to extend the AMR by at least 10-fold.

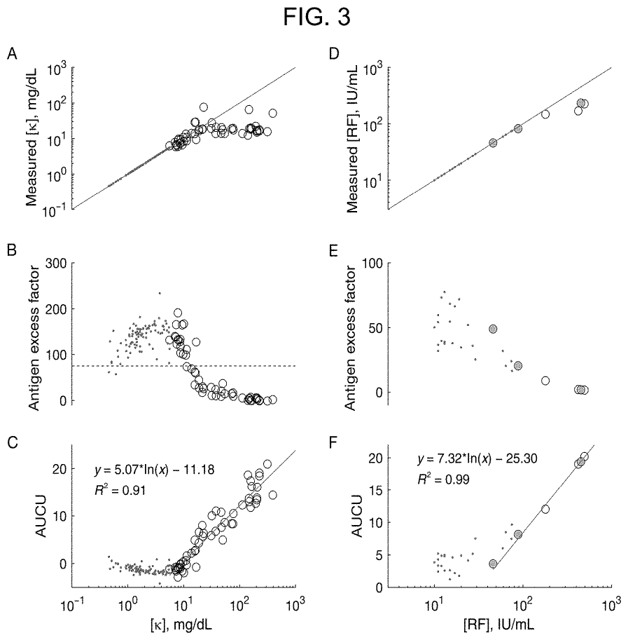

To test the utility of AUCU measurement in a clinical setting, kinetic data were collected for 150 routine κ sFLC measurements. For samples with concentrations within the manufacturer's measuring range (n=90; FIG. 3A-FIG. 3C, small closed symbols), the AUCU exhibited minimal variation around a slightly negative average value (−1.22±0.85). The Cobas instrument flagged 54 samples (36%) for values above linearity (n=43) or for abnormal kinetics (n=11) and automatically repeated these samples (FIG. 3A-FIG. 3C, large open symbols) after an additional 10-fold dilution. At <10 mg/dL, the concentrations measured on the initial and final dilutions were similar (FIG. 3A). At >10 mg/dL, the initial measurements generally underestimated the value measured on dilution, indicating that antigen excess was present. Consistent with this notion, the antigen excess factor, a previously reported method of kinetic analysis for antigen excess detection (7), DECREASED below the cutoff of 75, indicating likely antigen excess (FIG. 3B). As predicted by the kinetic model, the AUCU on the initial dilution increased with increasing κ sFLC concentrations >10 mg/dL, demonstrating a log-linear relationship with the concentration estimated on the final dilution (R²=0.91; FIG. 3C). Assuming that the estimate on higher dilution is more accurate because of the relief of antigen excess, these results demonstrate that AUCU provides a calibration curve in the zone of antigen excess.

To test the ability of the AUCU method to be used across immunoassays, the performance of the AUCU method was evaluated using kinetic data from 133 routine RF patient samples (FIG. 3D-FIG. 3F). Of these, 106 samples had RF activities below the measurable range and were excluded. Of the remaining samples, 23 had results within the linear range and acceptable kinetics (FIG. 3D-FIG. 3F, small closed symbols). For these samples, which were not repeated on dilution, the average AUCU was 5.23±3.82. The remaining 4 samples (3% of total) were flagged by the instrument and repeated on dilution owing to results greater than linearity (n=1) or abnormal kinetics (n=3). For these samples (FIG. 3D-FIG. 3F, large open symbols), the average AUCU was 17.7±3.79, significantly higher than the unflagged samples (P<0.0001 by 2-tailed t-test). To provide additional data points at intermediate concentrations, 2 additional dilutions of 1 of the high-value samples were prepared and analyzed (FIG. 3D-FIG. 3F, large shaded symbols). At <100 IU/mL, the initial measurements agreed well with those on dilution. At >100 IU/mL, the RF activities on the initial measurement were lower than those on dilution (FIG. 3D) and the antigen excess factor assumed a low value (FIG. 3E), suggesting antigen excess. As observed with κ sFLC data (FIG. 3C), the AUCU measured on the initial dilution demonstrated a log-linear relationship (R²=0.995) with the RF activities measured after dilution (FIG. 3F), suggesting that the AUCU on the first dilution could have been used to directly quantify RF activity in the zone of antigen excess.

### Discussion

Homogeneous turbidimetric immunoassays provide a fast, automated testing platform that can be readily adapted to different analytes. However, a major limitation of this assay format is the potential for nonlinearity caused by antigen excess. Because established methods for dealing with antigen excess require sample dilution, they compromise the very features that make the homogeneous immunoassay an attractive assay format: speed and automation. Each dilution adds additional reagent and operator costs, prolongs turnaround time, and introduces opportunities for laboratory errors. In this study, a hybrid computational/experimental approach was used to develop a new analysis method that could prevent sample dilutions. This approach uses the kinetic features of routinely collected data to accurately quantify analyte concentration despite antigen excess. Using clinical data from patients, we also show proof of principle that this new method can extend the AMR by at least 10-fold and thereby ameliorate the hook effect in 2 exemplary immunoassays. This new method should be generalizable to any turbidimetric or nephelometric immunoassay and, therefore, has the potential to extend the AMR for many immunoassays currently used in clinical laboratories.

Several previous studies have reported concentration-dependent changes in the kinetic features of the homogeneous immunoassay. Zuber and colleagues demonstrated that the duration of the initial delay in signal response, which immediately follows the addition of patient sample, becomes progressively shorter with increasing antigen concentration within the zone of antigen excess (11). Using a machine learning approach, Papik and colleagues defined an arbitrary classifier that successfully flagged antigen excess in ferritin measurements based on reaction kinetics (6). Finally, Urdal and colleagues designed and implemented a curvature-based kinetic flag by defining a ratio between the late and early absorbance changes in the sFLC assay (7). This flag successfully identified problematic specimens for subsequent dilution and repetition. Our work extends these earlier observations by deriving the AUCU and showing that it not only reports on the presence of antigen excess but also can provide a second calibration curve for use in the zone of antigen excess.

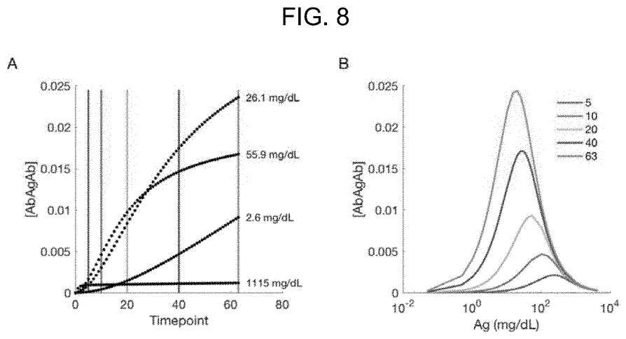

The major difference between the AUCU method proposed here and previous studies is that the AUCU method goes beyond simply flagging antigen excess and provides an opportunity to correctly quantify high antigen concentrations without sample dilution. On review of the literature, we found 2 reports that proposed methods to extend the AMR of the homogeneous immunoassay. First, Tarkkinen and colleagues showed that using an earlier time point to derive the end point calibration curve shifted the response curve toward higher concentrations, extending the upper limit of the AMR (9). The model developed in the present study can explain this phenomenon. At earlier time points, the model predicts that the degree of free Ab exhaustion will be less severe (FIG. 2A), diminishing the impact of antigen excess. Unfortunately, a large decrease in the end point [AbAgAb] accompanies the right shift in the dose-response curve (see FIG. 8), causing significant diminution of the signal magnitude and explaining why the application of the method of Tarkkinen et al. required specialized ultrasensitive instrumentation.

In a second study, Bicskei and colleagues (8) used curve fitting of a mechanistic model of a homogeneous immunoassay to directly measure antigen concentration in a clinical sample of unknown concentration. The major barrier to the implementation of this approach is that even the simplest mechanistic model, such as that developed in the current study, cannot be solved analytically. Without an analytical solution, model parameterization requires numerical simulation and sophisticated global fitting algorithms to efficiently search the parameter space. These methods require significant computing time, computing power, and technical expertise that may not be available in the clinical laboratory. In the present study, a practical solution is proposed by identifying a novel parameter, the AUCU, that provides a simple method for empirical calibration to the reaction curvature. To our knowledge, this is the first method that can extend the AMR using only the data that are routinely collected on a standard-issue chemistry analyzer, and simple algebra.

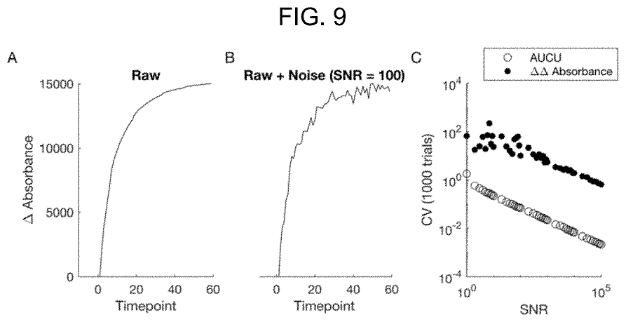

The major advantages of the AUCU over other methods of estimating reaction curvature are its robustness and its simplicity. Unlike point estimators of reaction curvature, such as a discrete second derivative or the ratio of late and early signal changes used by Urdal and colleagues (7), the AUCU provides a measure of the average curvature over the entire trace. This is an important advantage for 2 reasons. First, the curvature of the reaction kinetics changes with time and antigen concentration; therefore, it may be impossible to identify a single time point at which to calculate a point estimate of curvature at all antigen concentrations. Second, point estimates are more sensitive to noise in the recording because they do not average out random signal fluctuations, like the AUCU method. Indeed, in simulations directly comparing the AUCU method with the method of Urdal and colleagues, the AUCU method exhibited several log lower CV when gaussian noise was added to a real data trace (see FIG. 9). Another advantage of the AUCU method is that it provides a simple log-linear calibration curve rather than the inverse-asymptotic relationship of the alternative methodologies (FIG. 3). AUCU is computationally simple and can be calculated easily using kinetic data that are routinely collected by the Cobas c501 instrument; therefore, the AUCU method should have minimal technical barriers to implementation. As we demonstrated for 2 different immunoassays, the AUCU method appears to be robust and generalizable.

The AUCU method provides a calibration curve that functions only in the zone of antigen excess. Below this range, the AUCU assumes a constant low value. Therefore, it is important to determine when to use the AUCU calibration curve vs the standard end point calibration curve. One strategy can be to use the AUCU method only when the established antigen excess detection flags are tripped. However, the established flags are not able to detect all instances of antigen excess. Another strategy can be to use the AUCU value for both detection and correction of antigen excess by determining a threshold for the AUCU value above which the AUCU calibration curve should be used. A prospective validation study can compare the AUCU method with the existing modes of antigen excess detection.

The current study has demonstrated the use of the AUCU method in 2 different homogeneous turbidimetric immunoassays. Considering that 36% of the routine κ sFLC samples were repeated on dilution in the present study, it appears likely that adoption of the AUCU method could produce significant time and cost savings.

In summary, here is described a novel analysis methodology based on reaction kinetics that extends the AMR by at least 10-fold in 2 widely used clinical assays. This new approach has the potential to more readily detect and correct cases in which antigen excess produces falsely low (including false-negative) test results. This new method has the potential to be be readily deployed in clinical laboratories and may reduce costs by largely eliminating the need for repeat testing owing to antigen excess.

Nonstandard Abbreviations

AUCU: area under the curvature

AMR: analytical measurement range

sFLC: serum-free light chain

RF: rheumatoid factor

Ag: antigen

Ab: antibody

### REFERENCES

1. Heidelberger M, Kendall F E. A quantitative study of the precipitin reaction between type Ill pneumococcus polysaccharide and purified homologous antibody. J Exp Med 1929; 50:809-23.
2. Heidelberger M, Kendall F E. A quantitative theory of the precipitin reaction Ill. The reaction between crystalline egg albumin and its homologous antibody. J Exp Med 1935; 62:697-720.
3. Murata K, Clark R J, Lockington K S, Tostrud L J, Greipp P R, Katzmann J A. Sharply increased serum free light-chain concentrations after treatment for multiple myeloma. Clin Chem 2010; 56:16-8.
4. Vercammen M, Meirlaen P, Broodtaerts L, Vande Broek I, Bossuyt X. Effect of sample dilution on serum free light chain concentration by immunonephelometric assay. Clin Chim Acta 2011; 412:1798-804.
5. Jacobs J F M, van der Molen R G, Bossuyt X, Damoiseaux J. Antigen excess in modern immunoassays: to anticipate on the unexpected. Autoimmun Rev 2015; 14:160-7.
6. Papik K, Molnar B, Fedorcsak P, Schaefer R, Lang F, Sreter L, Tulassay Z. Automated prozone effect detection in ferritin homogeneous immunoassays using neural network classifiers. Clin Chem Lab Med 1999; 37: 471-6.
7. Urdal P, Amundsen E K, Toska K, Klingenberg O. Automated alarm to detect antigen excess in serum free immunoglobulin light chain kappa and lambda assays. Scand J Clin Lab Invest 2014; 74:575-81.
8. Bicskei Z, Ylander P, Hanninen P. Calibration of bioaffinity assays using kinetic data. J Biochem Biophys Methods 2006; 67:78-85.
9. Tarkkinen P, Palenius T, Lovgren T. Ultrarapid, ultrasensitive one-step kinetic immunoassay for Creactive protein (CRP) in whole blood samples: measurement of the entire CRP concentration range with a single sample dilution. Clin Chem 2002; 48: 269-77.
10. Holland, J. Adaptation in natural and artificial systems. Cambridge (MA): MIT Press; 1992.
11. Zuber E, Mathis G, Flandrois J P. Homogeneous two-site immunometric assay kinetics as a theoretical tool for data analysis. Anal Biochem 1997; 251:79-88.
12. Anderson, R. J., Studholme, R. M., Beckman Instruments, Inc. (1979). System for rate immunonephelometric analysis.

What is claimed is:

1. A method of reducing interference in an immunoassay, monitoring immunoassay reaction kinetics, detecting and correcting antigen excess, or extending the analytical measurement range (AMR) comprising:
generating, providing, or having been provided a target analyte concentration vs. time curve; and
measuring an area under the curvature (AUCU).

2. The method of claim 1, wherein the curve is generated by:
(i) providing or having been provided a sample comprising a target analyte;
(ii) contacting the sample comprising the target analyte with antibodies capable of crosslinking the target analyte to form an immune complex; and
(iii) detecting the target analyte and plotting the absorbance vs. time.

3. The method of claim 2, wherein the sample is a biological sample comprising a target analyte from a subject.

4. The method of claim 1, wherein the AUCU provides a log-linear calibration curve and increases proportionally to the target analyte concentration above a limit of an analytical measurement range (AMR) of a reaction endpoint.

5. The method of claim 1, wherein detecting the target analyte concentration comprises measuring absorbance or light scattering of the immune complexes.

6. The method of claim 1, wherein measuring the AUCU comprises:
(a) normalizing absorbance versus time data resulting in a normalized kinetic data function; and
(b) calculating the AUCU as a sum of the difference between the normalized kinetic data function and a line of unity, wherein the line of unity is the line resulting from the normalized absorbance at t=0 and t=t_{end}, wherein t_{end} is the time at the reaction endpoint.

7. The method of claim 1, wherein detecting the target analyte in the sample is performed using an automated chemistry analyzer to monitor a formation of light-scattering immune complexes that are generated when the target analyte cross-links a target analyte-specific reagent antibodies or antibody coated beads.

8. The method of claim 1, wherein the method of claim 1 is used if above the limit of the AMR, and a standard reaction endpoint calibration curve is used if below the limit of the AMR.

9. The method of claim 8, wherein a calibration curve choice is automated via a software tool.

10. The method of claim 1, wherein measuring the absorbance comprises measuring changes in light absorbance or light scattering.

11. The method of claim 1, wherein measuring absorbance vs. time is performed by recording, via a computer, kinetic data by monitoring the reaction at regular intervals prior to the reaction endpoint.

12. The method of claim 1, wherein the interference that is being reduced is the Hook effect.

13. The method of claim 1, wherein the immunoassay is a single-step homogeneous turbidometric or light absorbance assay.

14. The method of claim 1, wherein the immunoassay is a nephelometric or light scattering immune assay.

15. The method of claim 1, wherein sample dilution is not required if there is antigen excess.

16. The method of claim 1, wherein the AMR is extended by at least about 2-fold, at least about 3-fold, at least about 4-fold, at least about 5-fold, at least about 6-fold, at least about 7-fold, at least about 8-fold, at least about 9-fold, or at least about 10-fold.

17. The method of claim 1, wherein the AMR is extended by at least about 10-fold.

18. The method of claim 1, wherein the AUCU detects antigen excess.

19. The method of claim 1, wherein the AUCU provides a second calibration curve for use in a zone of antigen excess.

20. The method of claim 1, wherein the method quantifies high antigen concentrations without sample dilution.
